(19) United States
(12) Patent Application Publication
(10) Pub. No.: US 2003/0199856 A1
(43) Pub. Date: Oct. 23, 2003

(54) Coding system for the identification of tools

(75) Inventors: Uwe Hill, Remchingen (DE); Eberhard Korner, Bretten (DE); Steffen Konig, Ostringen (DE)

Correspondence Address: AKIN GUMP STRAUSS HAUER & FELD L.L.P., ONE COMMERCE SQUARE, 2005 MARKET STREET, SUITE 2200, PHILADELPHIA, PA, 19103-7013, US

(73) Assignee: Richard Wolf GmbH

(21) Appl. No.: 10/420,628

(22) Filed: Apr. 22, 2003

(30) Foreign Application Priority Data
Apr. 22, 2002 (DE) 102 17 811.9

(51) Int. Cl. A61B 17/00

(52) U.S. Cl. 606/1

(57) ABSTRACT

The invention relates to a coding system for the identification of tools in medical apparatus as well as to a corresponding method, with which successive code elements (16; 20) are attached on an outer side of a tool, and a stationary read means is arranged on a stationary part of an apparatus in the vicinity of the tool for detecting the successive code elements (16; 20) on movement of the tool (2) relative to the stationary part.

### BACKGROUND OF THE INVENTION

The invention relates to a coding system for the identification of rotating tools in medical apparatus as well as to a corresponding method for the identification of rotating tools in medical apparatus.

Medical apparatus with rotating tools are e.g. applied as tissue, cartilage and/or bone millers, such as for example are applied in arthroscopies for carrying out operative operations in body joints. These instruments consist essentially of a rotationally driven tool to be introduced into the joint cavity and a motor handle driving the tool. In the usual case, for carrying out surgical operations there are available a multitude of tool inserts which may be selectively connected to the motor handle. The different tools however often demand various operating parameters, such as for example tool rotational speed, so that a corresponding adjustment or activation of the motor is required.

There are already known systems for carrying out this adjustment with which a coding is provided on the tool and this is detected by a suitable sensor on the motor handle, in order for example to automatically adjust the rotational speed of the motor depending on the tool used. For this from U.S. Pat. No. 4,705,038, U.S. Pat. No. 5,217,478 and WO 97/16124 it is known to provide magnetic codings on the tools which are detected by a suitable sensor in the hand apparatus or motor handgrip. These codings are designed such that magnets, which are detected by reed or Hall sensors in the hand apparatus, are arranged in a stationary sleeve which surrounds the rotating tool. This stationary sleeve together with the tool is released from the hand part for exchange of the tool. The stationary sleeve together with the tool forms a unit, wherein the tool is rotatable in the sleeve. If the tool is connected to the hand part then the sleeve is held on the hand part in a rotationally fixed manner, whilst the tool may rotate driven by the motor. In this manner sensors such as reed sensors or Hall sensors arranged at certain positions in the hand part may detect whether magnets are arranged or not at corresponding, predefined locations in the sleeve. According to how many possible magnet positions and suitably arranged sensors are present, a limited number of different codes may be set on the tool for its identification.

This arrangement however has the disadvantage that for coding a large number of different tools a large number of sensors are required at different positions of the hand part. Furthermore the scope of information, which may be deposited in such a code, is limited so that beyond pure tool identification it is difficult to deposit further information in the coding provided on the tool. For example it may be desirable, apart from a pure identification of the tool to directly deposit the required motor rotation speed in coded form in the tool code. Furthermore it is conceivable to deposit further information such as the required rinsing flow etc. in this coding.

### BRIEF SUMMARY OF THE INVENTION

It is therefore the object of the invention to create a coding system as well as a method for identifying tools in medical apparatus, which permit a simplified coding with a large scope of information.

This object is achieved by a coding system with the features specified in claim 1 as well as by a method with the features specified in claim 11. Preferred embodiment forms are to be deduced from the dependent claims.

With the coding system according to the invention for the identification of tools in medical apparatus the coding is attached to the tool itself. This means the coding moves with the tool relative to a stationary part of the medical apparatus, which usually is a hand apparatus or motor handgrip.

Successive code elements are attached on a rotating tool preferably on an outer side in the circumferential direction, and on a stationary part of the apparatus in the proximity of the rotating tool there is arranged a stationary read means. In this manner, on rotation of the tool the code elements rotate relative to the stationary part of the apparatus or to the stationary read means which detects the code elements. At the same time the individual code elements successively move past the stationary read means and with this may be read out by this. At the same time the code represented by the code elements may be read out serially on passing the read means. Thus the rotation of the tool which is possible or takes place anyway is exploited in order to move the individual code elements in succession past the read means. It is thus possible to read out a very complex code with a large scope of information with only one sensor element in the form of the read means. This permits a considerably larger number of tools to be coded accordingly. Furthermore a larger information content of the coding may be achieved which allows also further operating parameters to be deposited in the code on the tool, for example the required rotational speed, the required quantity of rinsing fluid, etc. This data may be automatically read out by the read means and used for control of the motor or the drive in the stationary part of the apparatus. The scope of information, which may be deposited in the code, above all depends on the resolution of the read apparatus. With a large resolution capacity of the read apparatus the code elements may be formed correspondingly small so that a larger number of code elements may be arranged on a predefined distance section, for example the circumference of the tool.

Alternatively or additionally to the previously described embodiment form one may arrange the code elements in succession in the longitudinal direction of the tool and these may be detected with a movement of the tool in its longitudinal direction relative to the stationary part by the read apparatus. With this arrangement the code elements are likewise read out by a relative movement of the tool to a read means formed on the stationary part of the apparatus. However the reading-out in contrast to the previously mentioned embodiment example is not effected by rotation of the tool, but by a linear movement in the longitudinal direction of the tool. Thus code elements may for example be read out on application of the tool into a stationary part of the apparatus. For example the tool in its longitudinal direction is inserted into a corresponding receiver on the stationary part of the apparatus. At the same time a linear movement in the longitudinal direction of the tool is effected, by way of which the code elements are moved past the stationary read device in order to be able to be read out by the read means. With this embodiment too one preferably exploits the relative movement between the tool and the stationary apparatus part which is required for operation or application of the tool, in order to lead the code elements past a read means. The arrangement with which the code elements are arranged in succession in the longitudinal direction of the tool is not only suitable for rotating tools but also for stationary tools which are to be exchangeably arranged on a stationary apparatus part, for example on a grip part. Furthermore combinations of code elements arranged in the circumferential direction and in the longitudinal direction are also possible in order to make possible a more complex code with large information content.

Preferably the code elements comprise a multitude of strip-like elements parallel to one another which in the circumferential direction and/or the longitudinal direction of the tool are formed at different distances to one another and/or different widths on the surface of the tool for its identification. The code elements thus form a bar code as is basically known. For reading out, the individual strip-like elements in succession are moved past the stationary read means by the rotation or axial movement of the tool and are detected or read out by this. One may realise a certain number of different codes according to how fine the resolution of the strip or bar pattern is, in which the strip-like elements are arranged. Since such bar codes may be designed very finely, it is possible for a very large number of different tools be to coded accordingly. The strip-like elements are preferably arranged parallel to the rotational axis of the tool on its circumference so that they move past the read means on rotation in the circumferential direction, i.e. transverse to the direction of their extension.

In a further preferred embodiment form the code elements comprise a multitude of strip-like or circular segment shaped elements which are formed at differing distances to one another and/or different widths and are arranged along a circular plane in a plane transverse to the rotation axis of the tool on the tool. The type of coding and the reading-out is effected corresponding to the above described embodiment form. However with this second embodiment form the strip-like or circular segment shaped elements extend essentially radially to the rotation axis of the tool. The code elements are thus arranged annularly in a plane transverse to the rotation axis of the tool, for example on the end face of the tool. At the same time the read means is arranged such that here too the strip-like or circular segment shaped elements move relatively past the read means in the circumferential direction of the tool, i.e. essentially transversely to the direction of extension of the strip-like or circular segment shaped elements when the tool is rotated.

The code elements are furthermore preferably formed as webs. For this grooves or notches may be formed on the tool between which the code elements arise in the form of webs. It is accordingly also possible to provide projections on the tool. The read means at the same time is accordingly designed so that it may recognise whether material is present at a certain position or not, i.e. a read means detects the webs or the recesses between the webs.

The code elements may be formed of a sleeve or a disk which is fastened to the tool in a rotationally fixed manner. This permits the code elements to be manufactured independently of the tool and in manufacture to be subsequently connected to the tool. For example the code elements may be formed as webs in a sleeve-like or disk-like (sheet) plating. Punching may for example effect this, wherein the webs remain between the punched out recesses.

Preferably the tool comprises a stationary housing part and a moveable part arranged therein, wherein the code elements are arranged in the inside of the housing par. The stationary part is for example formed by a shank and a connecting coupling housing in whose inside there is arranged a rotatable or longitudinally displaceable shaft or a coupling. The stationary housing part, i.e. the coupling housing is connected to the stationary part of the apparatus, whilst the coupling and thus the shaft are connected to a drive means in the inside of the apparatus for moving the shaft. Preferably the code elements which are arranged on the moveable part of the tool are arranged such that they are located in the inside of the stationary housing part. Preferably the code elements are arranged in the region of the coupling in the inside of the coupling housing. By way of this the code elements are protected by the stationary housing part to the outside from contamination and damage. This is important with medical apparatus since these must be easy to clean and must be protected from contamination at locations which are difficult to clean. In particular if the code elements are designed in the form of recesses or projections, these are protected in the inside of the stationary housing part from contamination. A stationary read means is arranged on or in the stationary part of the apparatus. At the some time the read means may be arranged outside the stationary housing part of the tool and detect the code elements through the housing part or a walling of the housing part. For example the housing part may be designed in the form of a coupling housing or of a shank of plastic and a code element influencing a magnetic field may be detected through such a plastic walling. An optical detection is possible with a transparent design of the housing walling. Alternatively the stationary read means may project or engage into the stationary housing part of the tool.

It is further preferred for the code elements to be arranged in the inside of the moveable part of the tool. For example the code elements may be arranged in a coupling which is provided at the proximal end of the moveable part of the tool, for connection to a drive means. At the same time the code elements are preferably arranged in the inside of a hollow or sleeve-shaped coupling. The code elements may also be integrated into the material of the coupling, for example cast into this. Thus code elements of metal may be cast into a coupling of plastic. The detection of the code elements is effected as described previously, with this embodiment form too by way of a read means arranged on or in the stationary apparatus part, said read means being able to detect the code elements, e.g. optically or magnetically.

At the same time the code elements are preferably designed in a manner influencing a magnetic field and the read means detects changes of a magnetic field. The read means may for example be a Hall sensor which detects changes of the magnetic field. These changes of the magnetic field may be effected in that the code elements are designed in the form of notches or webs or projections which on passing the read means effect a change of the magnetic field which is detected by the read means. The read means is thus formed as a cogwheel sensor. Such a cogwheel sensor may for example consist of a Hall sensor in the known manner which is deposited on a permanent magnet. The changes of the magnetic field caused by the code elements are then detected via the sensor. Alternatively it is also conceivable to attach code elements of magnetic material on the tool which are detected by the read means. For this the read means may for example be designed as a reed contact.

Alternatively the code elements and the read means may be designed in a manner such that the code elements are optically detectable by the read means. For this for example code elements may be designed as reflecting elements on the tool. One may provide a bar code consisting of strips reflecting alternately better or worse, in particular reflecting and non-reflecting strips of a different width. The read means then acts in a manner such that the code elements are illuminated and a sensor detects the reflection or the strength of the reflection. In the simplest design for this the code elements may be designed as a bar code with alternatingly arranged black and white strips. It is however also possible to design the code elements in a disk as gaps or teeth which on rotation interrupt or let through a light beam which is detected by a suitable sensor. Common to all these various designs of code elements and read means is the fact that the code elements rotate together with the tool and are detected on passing the stationary read means so that the code formed by the code elements is read out serially. The read means then produces a corresponding signal which is conveyed further to the control means of the apparatus for the drive of the motor.

The code elements may represent a coded description of the tool type and/or of operating parameters for the tool. With the simplest form of the coding the code elements only identify a certain tool. The read means then reads out the coding and conveys this further to the control means which recognises the tool from the code and then carries out the control of the drive according to the applied tool. With this embodiment form it is necessary for the operating parameters which are required for certain tools to be deposited in the control or the apparatus and are called up after the associated tool is recognised. Alternatively it is also possible for the code on the tool itself to contain the required operating parameters in coded form. These are then read out by the read means and are conveyed further to the control means which then controls the apparatus and in particular the drive of the tool. This second embodiment form has the advantage that one may easily apply new tools without their operating parameters having to be previously given to the control means. The control means at the same time not only detects a certain tool type, but equally information as to which operating parameters need to be set.

The code elements may additionally contain position marking for determining the angular position and/or rotational speed of the tool. For this one may provide special code elements. Alternatively in particular for the detection of the rotational speed it is possible to do away with additional code elements, indeed only the repetition of any code pattern provided on the tool is detected and used for determining the rotational speed. As a whole this design has the advantage that one may do away with additional position and rotational speed sensors since this function may be assumed by the coding system. One may also provide a certain starter marking in order to mark the beginning of a code to be read out in order on rotation of the tool to give the read means information as to which location of the tool circumference or of the annularly arranged code the beginning of the code lies. The starter marking where appropriate may simultaneously be used for determining the position and rotational speed. The code elements may also be used for deactivating of drive means, for example if no tool is inserted into the handgrip.

The code elements preferably represent digital information. This is effected in that the code elements are arranged in a predefined raster. For example one may provide a strip division along the tool circumference. Each strip may have two condition of information, for example reflecting or non-reflecting. Alternatively the strip may be formed as a notch or web or projection or as a notch or solid material. Each strip may thus have two conditions which are detected by the read means. Thus a binary code may be represented in the coding. The read means detects this coding and emits a corresponding digital signal which may be processed further by a control means.

The invention further relates to a corresponding method for the identification of tools in medical apparatus. According to this method code elements are arranged on a certain tool in a predefined pattern for identification of the tool or for storing certain information. This information preferably concerns properties or operating parameters of the tool. For reading out the information the tool is moved relative to a stationary part of a medical apparatus, wherein the code elements pass a stationarily formed read means. The read means on movement of the tool detects the successive code elements and may read out the information deposited in a coded manner by way of the code elements and convey this further to a control means. With a multitude of tools which may be combined with a medical apparatus, e.g. hand apparatus, an individual code may be allocated to each tool. At the same time the method according to the invention permits a very large number of different tools to be coded accordingly. It is alternatively or additionally possible to deposit coded information directly on each tool according to the coding method according to the invention via operating parameters which are to be set. These may then be read out by the read means and transmitted further to a control device for controlling the apparatus.

According to a first preferred embodiment form of the method the code elements are arranged on a circular path concentric to the rotation axis of the tool and the tool is rotated relative to the stationary part of the apparatus for reading out. At the same time a relative rotation of the tool may be exploited which is required anyway for the intended drive of the tool, for example with a milling cutter or drill. In this manner a movement of the tool which is required anyway is exploited in order to produce the necessary relative movement between the code elements and the stationary read means for reading out the code elements.

Alternatively or additionally to this first embodiment form, the code elements may be arranged successively in the longitudinal direction of the tool and may be read out by the read means with a linear movement of the tool in its longitudinal direction relative to the stationary part of the apparatus. Such a linear movement of the tool in its longitudinal direction is effected for example on insertion and withdrawal of the tool from a stationary part of an apparatus, such as a handle for example. According to this embodiment form too a movement of the tool which is required in any case for operation of the tool is exploited in order to produce the necessary relative movement between the code elements and the stationary read means. It is furthermore possible to combine the linear and circumferential arrangement of code elements in order to be able to produce a more complex code and to be able to read out various codes with different movements of the tool. Furthermore the linear arrangement of the code elements in the longitudinal direction of the tool is also suitable for coding tools which do not rotate, and which is exchangeably attached to stationary apparatus parts.

### BRIEF DESCRIPTION OF THE DRAWINGS

Hereinafter the invention is described in more detail by way of example and by way of the accompanying figures. In these there are shown in

FIG. 1 a schematic sectional view of a tool with an associated read means,

FIG. 2 a coding sleeve according to a first embodiment example,

FIG. 3 a coding disk,

FIG. 4 a section view of a coding sleeve according to FIG. 2 with an associated signal course and

FIG. 5 a coding sleeve according to a second embodiment example.

### DETAILED DESCRIPTION OF THE INVENTION

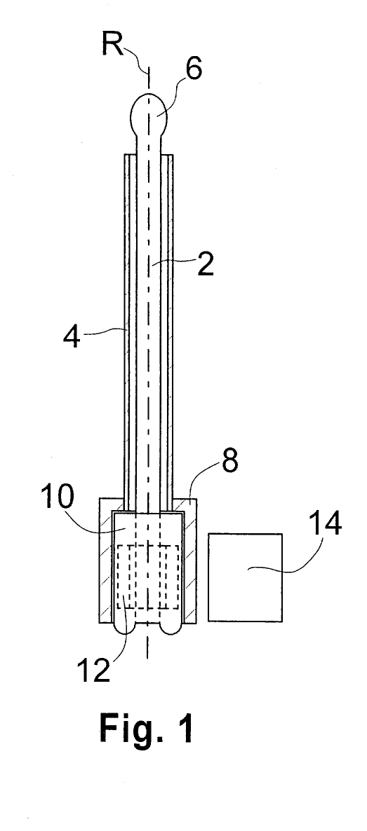

FIG. 1 shows a schematic sectional view of a tool with the coding system according to the invention. The tool consists of a rotatable shaft 2 which is arranged or guided in a stationary shank 4. On the upper end of the shank 1 in FIG. 1 the rotatable shaft 2 exits the shank 4 and forms the actual tool or the tool head 6 which carries out the machining. The shank 4 at the end which is opposite to the tool head 6 is connected to a coupling part 8 which in a manner not described here may be connected to a stationary apparatus part such as a hand apparatus or a motor handle. The rotatable shaft 2 likewise at its end opposite to the tool head 6, in the inside of the coupling part 8, comprises a coupling 10 for connection to the drive in the hand apparatus. The coupling 10 may for example be designed as a blade coupling. Since this design is already known a detailed description is not written here. In the region of the coupling 10 a coding sleeve or a target 12 is connected to the shaft 2 in a rotationally fixed manner and which circumferentially surrounds the shaft 2. The coding sleeve 12 comprises recesses distributed in the circumferential direction with webs formed between these of in each case a predefined width and predefined distance to one another which form the code elements. The code elements formed in the coding sleeve 12 in the form of recesses or webs of a certain width influence a magnetic field which is detected by a stationary read means 14. The read means 14 is arranged on the stationary apparatus part or the hand apparatus close to the accommodating tool.

The coding sleeve 12 is, as is shown in FIG. 1, arranged in the inside of the coupling 10. In this manner the coding sleeve is protected from contamination which may cling to the recesses or webs. The coupling housing or coupling part 8 connects to the stationary housing of the hand apparatus if the tool is applied onto a hand apparatus, so that the coupling 10 and the coding sleeve 12 arranged in its inside are arranged in a sealed-off space and thus additionally protected from contamination and damage. The coupling 10 in a rotationally fixed manner comes into engagement with a corresponding coupling of a drive in the hand apparatus. The read means 14 may detect the code elements formed on the coding sleeve through the walling of the coupling part 8 and the coupling 10. In order to allow this, the walling of the housing part 8 is preferably formed of plastic in order by way of the read means 14 to permit a disturbance-free detection of magnetic field changes which are produced on rotation by the coding sleeve 12 in the inside of the coupling.

If the tool is connected to the hand apparatus, the shaft 2 via the coupling 10 is driven by a drive in a manner such that it rotates about the rotation axis R. At the same time the individual code elements arranged behind one another in the circumferential direction in the coding sleeve 12 serially pass the read means 14 and are detected by this. The read means 14 emits a signal corresponding to the detected code elements which is processed further by a control apparatus for controlling the drive of the shaft 2.

This arrangement has the advantage that one requires only one sensor element in the form of the read means 14 for detecting a multitude of code elements. In this manner in a very simple way one may read out a complex code with a large content of information.

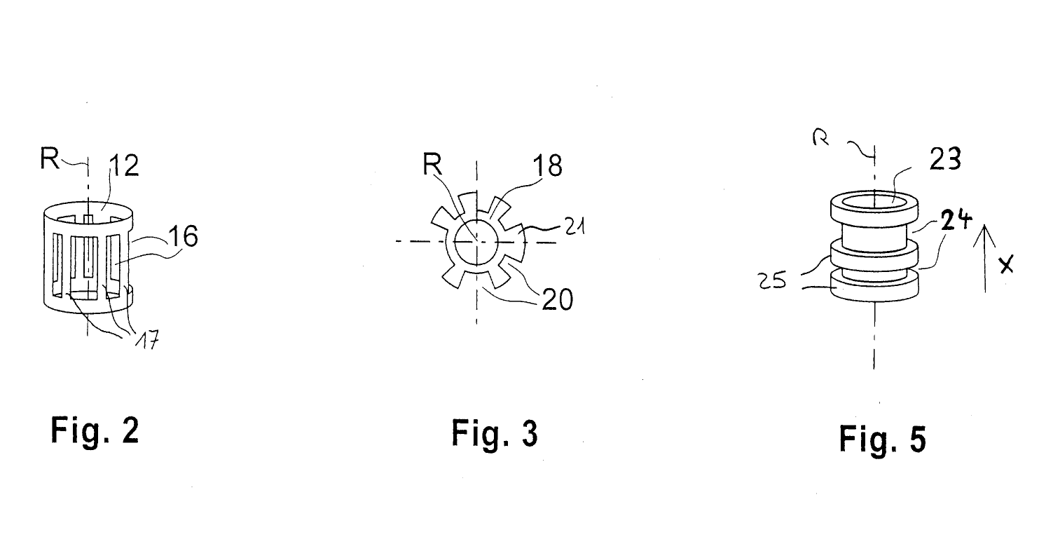

FIG. 2 shows a perspective detailed view of the coding sleeve 12. The coding sleeve 12 is formed annularly and is arranged concentrically to the rotation axis R on the shaft 2 in a rotationally fixed manner. In the coding sleeve 12 there are formed recesses 16 which are successive in the circumferential direction, with webs 17 lying between these. The webs 17 form code elements. At the same time the individual webs 17 may have widths varying in the circumferential direction and distances to one another varying in the circumferential direction in order to form various codings or codes in the coding sleeve 12. The code may be designed as a binary code 12. For this the circumference of the coding sleeve 12 is divided into strip-like sections, wherein each strip-like section may have two possible conditions. Either there is a web or a recess formed in the corresponding section. At the same time several recesses or web regions may directly follow one another. This has the effect that there arise recesses of a larger width or wider webs between individual recesses. The coding sleeve 12 forms a target influencing a magnetic field. The coupling 10 is preferably designed as a plastic coupling, and the read means 14 in the known manner may be designed as a cogwheel sensor. If now on rotation of the tool the individual recesses 16 or webs 17 pass the read means 14 serially or periodically, then the magnetic field changes serially or periodically which is detected by the read means. In this manner the read means may recognise whether at the corresponding position or the corresponding section of the coding sleeve 12 there is present a recess 16 or a web 17. At the same time, on rotation of the sleeve 12 the whole circumference of the sleeve 12 is scanned serially by the read means 14. A certain design of the recess 16 or of the web 17 or additional code elements may serve as a position marking. This on the one hand may serve for determining the starting position of the code formed along the circumference of the sleeve 12. Furthermore one or more position markings on the circumference may serve to determine the angular position of the shaft or its rotational speed. For this however one does not need to provide an additional position marking, but rather a certain location of the code serving the making of the tool or tool identification may be used for determining the position and rotational speed.

FIG. 3 shows an alternative embodiment form of the invention. With this the individual code elements are not formed in an annular sleeve, but on the circumferential edge of a circular disk 18. The disk 18 is connected to the shaft in a rotationally fixed manner and in a manner such that it extends transversally or normal to the rotation axis R. On the circumference or in the circumferential direction of the disk there are formed recesses 10 with webs lying therebetween, which form the code elements. According to the design of the coding sleeve 12 the circumference of the coding disk 18 may be divided into predefined segments which in each case have two conditions for the coding. Either there is provided a recess 20 or a web 21 in the region of a certain segment. If several segments with a recess follow one another, there arise recesses 20 with a larger width in the circumferential direction or webs 21 distanced further from one another, if two web segments follow one another. The disk 18 as also the sleeve 12 are preferably designed of a ferro-magnetic material so that the recesses 20 or webs 21 as well as the recesses 16 or webs 17 effect a change in the magnetic field on passing the read means 14, so that the read means 14 may detect whether at a position there is present a recess 16, 20 or a web 17, 21. Thus the reading out of the coding disk 18 is effected according to the reading out of the coding sleeve 12.

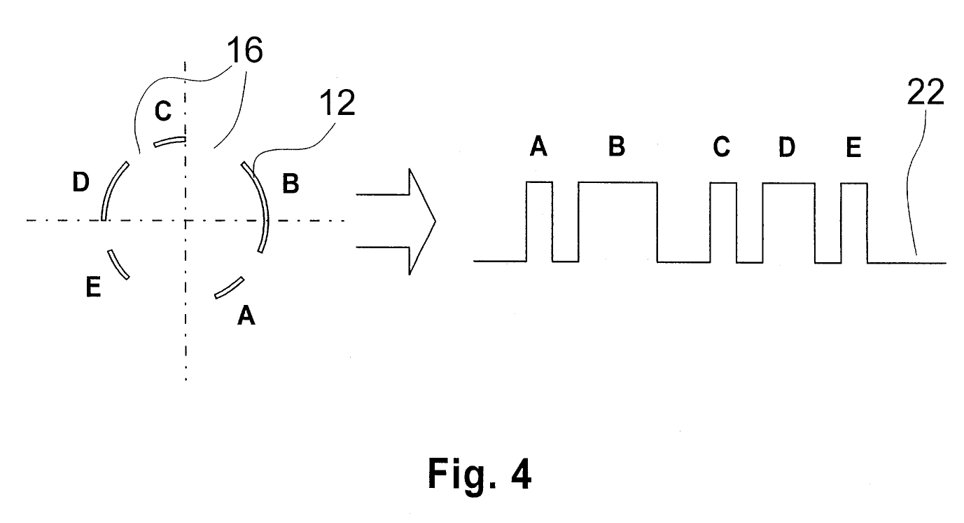

FIG. 4 shows a sectioned view through the coding sleeve according to FIG. 2. Between the recesses 16 there are located webs A to E of the sleeve 12. These webs A to E as also the recesses 16 have different widths in the circumferential direction, by which means one achieves a certain code. With another coding the recesses 16 and the webs A to E have other widths. According to the resolution capacity of the read means 14 the widths of the recesses 16 and webs A to E which are detectable in the circumferential direction may be selected very fine, by which means one achieves a large number of different codings. If the sleeve 12 passes the read means 14 on its rotation, the read means 14 detects the magnetic field changes effected by the recesses 16 or the webs A to E lying therebetween and emits the corresponding signal course 22 which is shown in FIG. 4 on the right. This signal course 22 is a digital signal which may be processed further by a control means.

FIG. 5 shows a second embodiment form of a coding sleeve 23 with which the code elements in the form of recesses 24 and webs 25 are arranged in the linear direction in the direction of the axis R. The design of the coding and also the reading-out corresponds essentially to the designs explained by way of FIGS. 2 and 3, with which the code elements are arranged in the circumferential direction or on a circular line. With the embodiment form according to FIG. 5 the reading out of the code is not effected by rotation of the tool relative to a stationary apparatus part such as a handgrip, but by way of axial linear movement in the direction X parallel to the axis R. Such a movement is for example effected on insertion or removal of the tool from a handgrip. This design is also suitable for stationary tools which are not to rotate. Furthermore a combination of the coding according to FIG. 5 with one of the examples explained by way of FIGS. 2 and 3 is possible. With a combination with the embodiment example according to FIG. 2 one may arrange the webs 17 according to FIG. 2 instead of the inner walling holding together the webs 25. With this arrangement then the webs 17 and 25 are arranged crossed to one another at right angles. With a combination of embodiment examples according to FIGS. 3 and 5 the end face of the disk 18 which faces the axis R may for example be provided with webs 25 according to FIG. 5. These combinations of the various codings on the one hand permit a more complex code for the design, with a larger number of codings and on the other hand it is possible to read out various codes with different movement directions of the tool.

Instead of merely providing recesses or webs in the sleeve 12 or the disk 18, the individual code elements may be designed such that it provides more than two possible conditions of the magnetic field. In this manner one may accommodate an even greater quantity of information in the coding, wherein a suitable analog signal is then emitted by the read means 14. The simplest form of the digital coding shown here in the sleeve 12 or disk 18 recognises only two conditions, specifically “material” or “no material”. Accordingly the coding may for example also be designed optically in that the two conditions for example indicate “reflecting” or non-reflecting” which may be detected by a suitable optic sensor. The basic concept of the invention however remains the same for all possible and known design forms of the code elements, that the code elements rotate together with a tool and are serially scanned or read out by a stationary sensor.

LIST OF REFERENCE NUMERALS

| | |
|---|---|
| 2 | shaft |
| 4 | shank |
| 6 | tool head |
| 8 | coupling part |
| 10 | coupling |
| 12 | sleeve |
| 14 | read means |
| 16 | recess |
| 17 | web |
| 18 | disk |
| 20 | recess |
| 21 | web |
| 22 | signal course |
| 23 | sleeve |
| 24 | recess |
| 25 | web |
| R | rotation axis |
| A to E | webs |
| X | movement direction |

1. A coding system for the identification of tools (2) in medical apparatus, with which successive code elements (16; 20; 24) are brought on an outer side of a tool (2), and a stationary read means (14) is arranged on a stationary part of an apparatus in the vicinity of the tool for detecting the successive code elements (16; 20; 24) on movement of the tool (2) relative to the stationary part.

2 A coding system according to claim 1, with which the tool (2) is a rotating tool, the code elements (16; 20) are arranged in succession in the circumferential direction and may be detected by the read means (14) on rotation of the tool (2) relative to the stationary part.

3. A coding system according to claim 1 or 2, with which the code elements (24) are arranged in succession in the longitudinal direction of the tool (2) and may be detected by the read means (14) with a movement of the tool (2) in its longitudinal direction relative to the stationary part.

4. A coding system according to one of the preceding claims, with which the code elements (16; 24) comprise a multitude of strip-like elements parallel to one another, which in the circumferential direction and/or longitudinal direction of the tool (2) are formed with different distances to one another and/or different widths on the surface of the tool (2) for its identification.

5. A coding system according to one of the preceding claims, with which the code elements (20) comprise a multitude of strip-like or circular segment shaped elements (20) which are formed with different distances to one another and/or with different widths, and are arranged along a circular line in a plane transverse to the rotation axis (R) of the tool (2) on the tool (2).

6. A coding system according to one of the preceding claims, with which the code elements (16; 20; 24) are formed as webs (17; 21; 25).

7. A coding system according to one of the preceding claims, with which the code elements (16; 20; 24) are formed on a sleeve (12; 23) or disk (18) which is connected to the tool (2) in a rotationally fixed manner.

8. A coding system according to one of the preceding claims, with which the tool
comprises a stationary housing part (4; 8) and a moveable part (2) arranged therein, wherein the code elements (16; 20; 24) are arranged in the inside of the housing part (4;8).

9. A coding system according to claim 8, with which the code elements (16;20;24) are
arranged in the inside of the moveable part (2).

10. A coding system according to one of the preceding claims, with which the code elements (16; 20; 24) are designed influencing a magnetic field or are optically detectable.

11. A method for the identification of tools (2) in medical apparatus, with which successive code elements (16; 20; 24) are arranged on a tool (2) which indicate information concerning the tool (2), and for reading out the information, the tool (2) is moved relative to a stationary part of an apparatus, wherein the code elements (16; 20; 24) pass a stationarily formed read means (14) which detects the code elements (16; 20; 24) for reading out the information.

12. A method according to claim 10, with which the code elements (16; 20) are arranged on a circular path concentric to the rotational axis (R) of the tool (2) and for reading out, the tool (2) is rotated relative to the stationary part of an apparatus.

13. A method according to claim 10 or 11, with which the code elements (24) are arranged in succession in the longitudinal direction of the tool (2) and with a linear movement of the tool (2) in its longitudinal direction relative to the stationary part of an apparatus are read out by the read means (14).
